(19) United States
(12) Patent Application Publication
(10) Pub. No.: US 2026/0026696 A1
(43) Pub. Date: Jan. 29, 2026

(54) A MONITORING DEVICE

(71) Applicant: CERYX MEDICAL LIMITED, Cardiff, Wales (GB)

(72) Inventors: Ashok Singh CHAUHAN, Cardiff, Wales (GB); Graham John FISHER, Cardiff, Wales (GB)

(21) Appl. No.: 19/099,784

(22) PCT Filed: Aug. 1, 2023
(86) PCT No.: PCT/GB2023/052027

§ 371 Date: Jan. 30, 2025

(30) Foreign Application Priority Data
Aug. 1, 2022 (GB) 2211188.4

(51) Int. Cl. A61B 5/0205; A61B 5/00; A61B 5/024; A61B 5/08; A61B 5/11; A61N 1/365

(52) U.S. Cl. CPC A61B 5/0205; A61B 5/02405; A61B 5/0816; A61B 5/1118; A61B 5/7275; A61B 5/7435; A61N 1/36521; A61B 2560/0271

(57) ABSTRACT

A cardiorespiratory monitoring device, configured to: receive sensor data comprising cardiac data and respiratory data for a subject; determine a respiratory sinus arrhythmia, RSA, value based on the cardiac data and the respiratory data: determine a cardio-respiratory phase synchronisation, CRPS, value based on the cardiac data and the respiratory data; and output a health indication for the subject based on the RSA value and the CRPS value.

### FIELD

The present disclosure relates to a cardiorespiratory monitoring device, and in particular, although not exclusively, to a controller for a cardiorespiratory monitor for monitoring the cardiorespiratory health of a subject.

### BACKGROUND

Monitoring devices for monitoring the health of a patient or other subject's heart and other physiological functions are commonly known in the art. Monitoring devices, can include: one or more sensors for sensing physiological data relating to the subject. The one or more sensors of a monitoring device may include a cardiac sensor. The cardiac sensor may comprise an electrode arrangement comprising cardiac electrodes which can be positioned in, on or about the heart to sense the cardiac electrical activity of one or more heart chambers. Some devices may have heart pacing functionality and the electrodes may also be used to additionally provide a pacing signal to the heart. The electrodes may comprise wires implanted into the heart, for example, in the right ventricle or the left ventricle.

The sensors of a monitoring device may also include a respiratory sensor. For example, a metabolic demand of a patient may be determined by monitoring the respiration of the patient. The respiratory sensor may comprise one or more of: an air flow sensor, a motion sensor, such as a thoracic abdominal motion sensor, a transthoracic impedance sensor comprising electrodes placed on the abdomen, a visual sensor for visualising movement of the abdomen or a strain gauge placed on the patient's chest. Respiratory data may also be determined by computer analysis of arterial waveforms. The respiratory data may include respiratory cycles determined from zero-crossings of the transthoracic impedance.

Monitoring devices may also receive activity data from an activity sensor. The activity sensor may comprise the cardiac sensor and/or the respiratory sensor. For example, activity data may be determined by monitoring the heart rate or respiratory rate of the patient. The activity sensor may also comprise one or more other sensors such as accelerometers, pulse oximeters, perspiration sensors, video sensors, temperature sensors or other sensors as known in the art.

Monitoring devices may output cardiac data for monitoring heart rate and the presence of cardiac events over an extended period of time. Monitoring devices may output respiratory data for monitoring respiratory rate and events like sleep apnia, asthma etc over an extended period of time. Cardiac data and respiratory data may be referenced against activity level to segment periods when the patient is sleeping, at rest, moderately active, exercising etc.

### SUMMARY

According to a first aspect of the present disclosure there is provided a cardiorespiratory monitoring device, configured to:
receive sensor data comprising cardiac data and respiratory data for a subject;
determine a respiratory sinus arrhythmia, RSA, value based on the cardiac data and the respiratory data;
determine a cardio-respiratory phase synchronisation, CRPS, value based on the cardiac data and the respiratory data; and
output a health indication for the subject based on the RSA value and the CRPS value.

The cardiac data and the respiratory data may be captured over a fixed or predetermined time period. The health indication may be a combined health indicator for the RSA and CRPS values.

The device may be configured to:
determine a subject activity level based on the sensor data; and
   determine the RSA value based on at least a portion of the cardiac data and the respiratory data for which the subject activity level is less than a RSA threshold activity level; and/or
   determine the CRPS value based on at least a portion of the cardiac data and the respiratory data for which the subject activity level is greater than a CRPS threshold activity level.

The device may be configured to:
receive activity data;
determine a plurality of RSA values and a plurality of CRPS values during a period of physiological demand as indicated by the activity data; and
determine the health indication based on the plurality of RSA values and the plurality of CRPS values.

The device may be configured to determine the health indication based on:
a variation in the RSA values during the period of physiological demand; and
a variation in the CRPS values during the period of physiological demand.

The variation in CRPS values may define a duration of phase locking.

The period of physiological demand may comprise: a period of exercise; or a period of brain activity.

The sensor data may further comprises one or more of: cardiological data, pulse oximeter data, accelerometer data, body temperature data, perspiration data, camera data, an adenosine triphosphate, ATP, level and an adrenaline level.

The device may be configured to:
identify a plurality of respiratory cycles from the respiratory data;
identify a plurality of heart beats from the cardiac data;
associate each heart beat with a respiratory cycle and a respiratory phase angle of the respiratory cycle to provide labelled cardiorespiratory data;
determine the CRPS value from the labelled cardiorespiratory data.

The device may be configured to:
determine the CRPS value based on a variance in respiratory phase angles over the plurality of respiratory cycles.

The device may be configured to:
determine the CRPS value based on a variance in heart beats per respiratory cycle.

The device may be configured to:
identify one or more respiratory cycles from the respiratory data;
identify a plurality of heartbeats from the cardiac data;
identify one or more inspiration sequences of heart beats in inspiration phases of the plurality of respiratory cycles and one or more of expiration sequences of heart beats in expiration phases of the plurality of respiratory cycles; and
determine the RSA value based on an inspiration heart rate of the one or more inspiration sequences and an expiration heart rate of the one or more expiration sequences.

The device may be configured to determine the RSA value as a ratio of the inspiration rate to the expiration rate.

The device may be configured to:
determine a RSA health factor for the subject by comparing the RSA value to one or more RSA reference values;
determine a CRPS health factor for the subject by comparing the CRPS value to one or more CRPS reference values; and
output the health indication based on the RSA health factor and the CRPS health factor.

The RSA health factor may be determined in a first time period. The CRPS health factor may be determined in a second time period. The first time period may be different from the second time period. The first time period may be continuous or discontinuous. The second time period may be continuous or discontinuous.

The device may be configured to determine the health indication using a look up table and the CRPS value and the RSA value.

The sensor data may comprise a plurality of sensor readings over a time window and wherein the device is configured to determine a plurality of RSA values and a plurality of CRPS values and output the health indication based on the plurality of RSA values and the plurality of CRPS values.

The device may be configured to:
determine a RSA health factor for the subject patient by comparing the plurality of RSA values to one or more RSA reference values;
determine a CRPS health factor for the subject patient by comparing the plurality of CRPS values to one or more CRPS reference values; and
output the health indication based on the RSA health factor and the CRPS health factor.

The device may be configured to output the health indication to one or more of:
a subject user interface;
a clinician device;
a pacemaker device;
a peripheral stimulation device;
an emergency responder device; and
a remote monitoring centre.

The device may be configured to output the health indication for display. The device may be configured to output the health indication for display on a user interface of a patient device and/or a clinician device.

The device may be configured to output therapeutic instructions to a subject user interface if the health indication is representative of cardiorespiratory stress in the subject.

The therapeutic instructions may comprise one or more of:
breathing instructions;
activity instructions; and
an instruction to seek immediate medical attention.

According to a second aspect of the present disclosure, there is provided a cardiorespiratory monitoring device, configured to:
receive sensor data comprising cardiac data and respiratory data for a subject;
determine cardiorespiratory synchronisation data based on the cardiac data and the respiratory data;
determine a cardiorespiratory coupling factor based on a response of the cardiorespiratory synchronisation data to a period of physiological demand as indicated by the sensor data; and
output a health indication for the subject based on the cardiorespiratory coupling factor.

The sensor data may comprise activity data. Activity data may be derivable from the cardiac data or respiratory data.

The device may determine the cardiorespiratory coupling factor based on a response of the cardiorespiratory synchronisation data to a period of physiological demand as indicated by the activity data.

The device may be configured to determine the cardiorespiratory synchronisation data by determining a plurality of respiratory sinus arrhythmia, RSA, values based on the cardiac data and the respiratory data.

The device may be configured to determine the cardiorespiratory coupling factor based on a variation in the plurality of RSA values during the period of physiological demand.

The device may be configured to determine the cardiorespiratory synchronisation data by determining a plurality of cardio-respiratory phase synchronisation, CRPS, values based on the cardiac data and the respiratory data.

The device may be configured to determine the cardiorespiratory coupling factor based on a variation in the plurality of RSA values during the period of physiological demand.

The period of physiological demand may comprise: a period of exercise; or a period of brain activity above a brain activity threshold.

The device may be configured to output the health indication to one or more of:
a subject user interface;
a clinician device;
a pacemaker device;
a peripheral stimulation device;
an emergency responder device; and
a remote monitoring centre.

The device may be configured to output the health indication for display. The device may be configured to output the health indication for display on a user interface of a patient device and/or a clinician device.

According to a third aspect of the present disclosure there may be provided a cardiorespiratory monitoring system comprising:
any device disclosed herein; and
one or more sensors configured to provide the sensor data.

The cardiorespiratory monitoring system may further comprise a patient user interface for displaying the health indication.

According to a fourth aspect of the present disclosure there is provided a method for providing a health indication, the method comprising:
receiving sensor data comprising cardiac data and respiratory data for a subject;
determining a respiratory sinus arrhythmia, RSA, value based on the cardiac data and the respiratory data;
determining a cardio-respiratory phase synchronisation, CRPS, value based on the cardiac data and the respiratory data; and
outputting a health indication for the patient based on the RSA value and the CRPS value.

According to a fifth aspect of the present disclosure there is provided a method for providing a health indication, the method comprising:
receiving sensor data comprising cardiac data and respiratory data for a subject;
determining cardiorespiratory synchronisation data based on the cardiac data and the respiratory data;
determining a cardiorespiratory coupling factor based on a response of the cardiorespiratory synchronisation data to a period of physiological demand as indicated by the sensor data; and
outputting a health indication for the subject based on the cardiorespiratory coupling factor.

According to a sixth aspect of the present disclosure there is provided a computer program product storing executable instructions for performing any method disclosed herein.

According to a further aspect of the present disclosure there is provided a cardiorespiratory monitoring device, configured to:
receive sensor data comprising cardiac data and respiratory data for a subject;
determine a respiratory sinus arrhythmia, RSA, value based on the cardiac data and the respiratory data;
determine a phase locking value based on the cardiac data and the respiratory data; and
output a health indication for the subject based on the RSA value and the phase locking value.

The phase locking value may comprise an inspiration onset phase angle and/or an expiration onset phase angle.

The phase locking value may provide a phase synchronisation value when RSA is present.

The disclosed devices, systems and methods can monitor the RSA and CRPS synchronisation and determine the strength of the existing RSA and CRPS synchronisations. RSA synchronisation can be monitored when the subject is at rest and CRPS synchronisation can be monitored when the subject is physically active. Both RSA and CRPS synchronisations can be monitored in subjects that are moderately active.

A strength of synchronisation can be estimated for example by amplitude of RSA and/or phase locking in CRPS. A healthy cardiorespiratory system can show stronger/long term synchronisation as compared to a not so healthy system.

There may be provided a diagnostic device, configured to:
receive sensor data comprising respiratory data and receive sensor data comprising heart-beat/cardiac data;
determine RSA value from the data;
determine CRPS value from the data; and
   compute a coupling factor based on RSA value;
   compute a coupling factor based on CRPS value; and/or
   compute a coupling factor based on RSA and CRPS value.

The disclosed devices, systems and methods can determine a healthy cardiorespiratory system from unhealthy system based on the coupling factor value.

The disclosed devices, systems and methods can determine the severity of weak coupling and irregular cardiorespiratory output for hospital monitoring, medical intervention, etc.

The disclosed devices, systems and methods can provide for derivation of a metric indicating current healthiness of cardiovascular metabolism based on phase locking of RSA and CRPS modes to respiration. This could be additional to already known metric calculations such as blood oxygen level, minute ventilation etc.

The disclosed devices, systems and methods can combine the phase locking with or without RSA amplitude at low level of exercise to derive a metric of health and continue to derive metric at high exercise levels when RSA fades away while CRPS starts to emerge and dominate with increasing exercise levels. The disclosed devices, systems and methods can use CRPS as a valid diagnostic mechanism at high level of exercise. This metric could be calculated for either RSA or CRPS, or for both combined in some proportions uniquely offering assessment mechanism from rest to high exertion levels.

Diagnostic/prognostic/monitoring device can analyse coupling factor K based on the heart-lung synchronisation and, including but not limited to, physiological variables such as cardiac output, blood pressure, heart rate, partial oxygen pressure.

Coupling factor, K reflects the responsiveness of heart to physiological demands including but not limited to physical constraints, brain activities such as autonomic nervous system response,

Coupling factor is high in a healthy cardiorespiratory system and decreases with cardiorespiratory illness, ageing and other health related factors.

Estimate of coupling factor may be estimated for either RSA, CRPS or both.

Coupling factor calculation can include,
1. in RSA mode on the basis of; either one or more of the variables combined, i.e., RSA amplitude, average number of heartbeats per inspiration and expiration period, and phase-locking to respiration cycles.
2. in CRPS mode on the basis of phase-locking and/or average number of heart beats per inspiration and expiration period in CRPS mode.
3. in any mode, the duration for which synchronisation could be maintained.

Diagnostic/prognostic/monitoring device can provide the capability of indirect controlling of heart-lung synchronisation as suggested by the device, such as by directed breathing, transcutaneous stimulation of nerves.

A version of diagnostic/prognostic/monitoring device could be designed as invasive or non-invasive device to control the heart-lung synchronisation.

While the disclosure is amenable to various modifications and alternative forms, specifics thereof have been shown by way of example in the drawings and will be described in detail. It should be understood, however, that other embodiments, beyond the particular embodiments described, are possible as well. All modifications, equivalents, and alternative embodiments falling within the spirit and scope of the appended claims are covered as well.

The above discussion is not intended to represent every example embodiment or every implementation within the scope of the current or future Claim sets. The figures and Detailed Description that follow also exemplify various example embodiments. Various example embodiments may be more completely understood in consideration of the following Detailed Description in connection with the accompanying Drawings.

### BRIEF DESCRIPTION OF THE DRAWINGS

One or more embodiments will now be described by way of example only with reference to the accompanying drawings in which:

FIG. 1A illustrates a plot of respiratory sinus arrhythmia (RSA) amplitude against time for a healthy individual prior to, during and subsequent to physical exercise;

FIG. 1B illustrates a plot of RSA amplitude against time for an elderly individual prior to, during and subsequent to physical exercise

FIG. 2A illustrates a synchrogram plot corresponding to the RSA plot of FIGS. 1A;

FIG. 2B illustrates a synchrogram plot corresponding to the RSA plots of FIG. 1B;

FIG. 2C illustrates simulated cardio-respiratory data for a healthy heart exhibiting RSA and an unhealthy heart exhibiting no RSA;

FIG. 2D illustrates simulated cardio-respiratory data for an unhealthy heart that does not exhibit RSA;

FIG. 3 illustrates a schematic block diagram of a cardiorespiratory monitoring system;

FIG. 4 illustrates a plot of pulse sequences against time for two successive heart cycles; and

FIG. 5 illustrates a method for providing cardiorespiratory monitoring for a patient; and

FIG. 6 illustrates example cardiac data and respiratory data in a subject exhibiting CRPS but not RSA.

### DETAILED DESCRIPTION

The present disclosure relates to a cardiorespiratory monitoring device that provides an indication of patient health based on the synchronisation between monitored cardiac data and respiratory data. The method of monitoring and health indication is derived from the inventor's observations of natural pacing in both healthy subjects and those with a range of physiological conditions under various levels of physical exertion. The proposed cardiorespiratory monitoring scheme aims to monitor the cardiorespiratory data over varying degrees of physical demand on the subject with reference to the natural pacing observed in healthy subjects in such conditions.

The body's natural regulation of heartbeat is in phase with the breathing or respiratory cycle. Synchronisation between the heart and lungs is indicative of a healthy cardiorespiratory system. Coupling strength between the heart and lungs is one of the key factors behind increased synchronisation and decreased coupling strength—for instance through ageing and in patients with heart failure and other cardiorespiratory disorders-results in decreased synchronisation between the heart and lungs.

One example of synchronisation between the heart and lungs includes a reduction in heart rate during expiration (breathing out) and an increase in heart rate during inspiration (breathing in). This is referred to as respiratory sinus arrhythmia (RSA). The loss of RSA is a predictor of cardiovascular risk and a prognostic indicator for multiple diseases including heart failure.

Example pacing systems can be provided in order to artificially restore, maintain or enhance RSA to obtain a profile of a healthy individual. Applications GB 1913050.9 and PCT/GB2020/052149 disclose such systems and are hereby incorporated by reference in their entirety. Example systems may control the timing of stimulus signals (the pacing signal) according to a non-linear function. The non-linear function may be generated by a neuronal oscillator provided by a central pattern generator or a digital simulation.

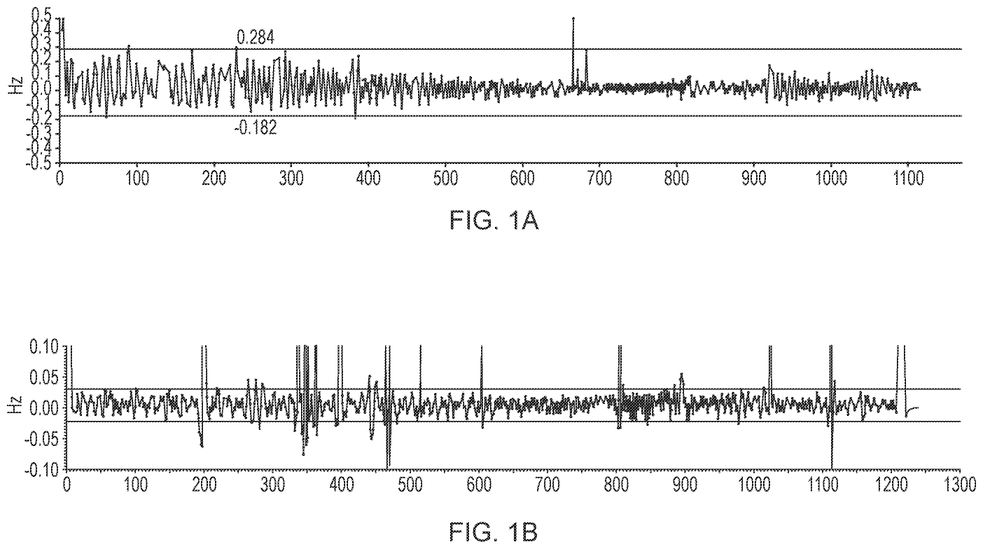

FIGS. 1A and 1B illustrate plots of RSA amplitude against time for respective first and second individuals prior to, during and subsequent to physical exercise. The data for FIG. 1A was acquired from a healthy individual (age 24+, height 180.5 cms, weight 88 Kg, BMI 27.0) while the data for FIG. 1B was acquired from an elderly, otherwise healthy, individual (age 64+, height 178 cms, weight 93 Kg, BMI 29.4). In both figures, the subjects are at rest at time=0 seconds. In FIG. 1A, exercise onset occurs at time=180 seconds and the exercise period ends at time=800 seconds. In FIG. 1B, exercise onset occurs at time=300 seconds and the exercise period ends at time=800 seconds

FIG. 1A indicates a healthy RSA level of ˜ 0.5 Hz peak to peak amplitude for the first individual at rest. The first individual was subject to a gradually increasing intensity of exercise from 180s to 800s. The RSA amplitude remains high before the onset of exercise (i.e. before 180s) and during the initial phase of exercise (i.e. between 180s and 400s). The RSA amplitude decreases, to less than 0.1 Hz, at higher intensity of exercise (i.e. between 500s and 800s). In the recovery time (i.e. after 800s), the RSA amplitude starts increasing in amplitude again.

FIG. 1B indicates an unhealthy level of RSA of ˜ 0.05 Hz peak to peak amplitude for the second individual at rest. As seen in the figure, the RSA amplitude also decreases following exercise onset and recovers again following the end of exercise. The trend in FIG. 1B is less obvious than that in FIG. 1A because the resting level of RSA is relatively low to begin with so there is a high signal to noise ratio,

FIGS. 1A and 1B illustrate that RSA decreases during physical activity for individuals with both healthy and unhealthy cardiorespiratory systems. RSA amplitude is high in healthy subjects at rest and decreases with increased exertion levels. Individuals with an unhealthy cardiorespiratory system tend to have lower RSA amplitude, even at rest, which further decreases with increased exertion.

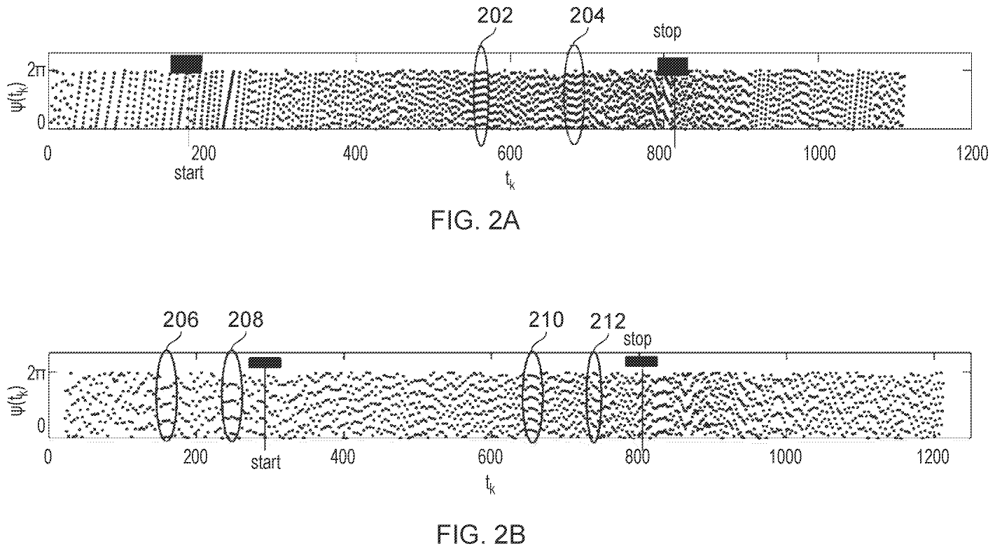

FIGS. 2A and 2B illustrate synchrogram plots corresponding to the respective RSA plots of FIGS. 1A and 1B. A synchrogram plots heartbeat instances, in time, on the x-axis with respect to a respiratory phase angle on the y-axis. Here, respiratory phase angle defines an instantaneous phase angle of the respiratory cycle, relative to the start of the respiratory cycle. The respiratory phase varies or cycles between 0 and 2π radians during a single respiratory cycle in the same way as a conventional oscillating system.

For the first individual (FIG. 2A), a regular pattern of heartbeats with approximately 12 heartbeats per respiratory cycle can be seen during the period prior to exercise onset at 180 seconds. This regular heart rate and a high number of heartbeats per respiratory cycle correspond to the period of high amplitude RSA. As the exercise progresses and the RSA amplitude decreases a second type of synchronisation becomes identifiable in the synchrogram plot-cardio-respiratory phase synchronisation (CRPS). CRPS may also be referred to herein as phase locking. Regions of CRPS 202, 204 can be seen in the synchrogram plot as regions in which the respiratory phase angle of corresponding heartbeats in adjacent respiratory cycles have a substantially constant phase angle. In other words, the heart beats or pulses at the same repeating phase angles in consecutive respiratory cycles. The regions of CRPS 202, 204 can be identified in the synchrogram plot as substantially horizontal lines or plateaus formed by the heartbeat instances in adjacent respiratory cycles. CRPS is not necessarily limited to heart beats having the same phase angles in consecutive or adjacent respiratory cycles and that the repeat sequence of phase angles may extend over an integer number of respiratory cycles (for example, up to one of 2, 3, 4, 6 or more respiratory cycles). As explained below in relation to FIGS. 2C and 2D, CRPS tends to be continually present subject to interruptions in the respiratory cycle due to ingestion, yawning, hiccups etc. Therefore, FIG. 2A includes regions of CRPS that are not immediately identifiable because the heart rate is relatively low (for example prior to exercise onset) and/or because the repeat sequence of phase angles is over a number of respiratory cycles.

For the second individual (FIG. 2B), there are identifiable regions of CRPS 206, 208, 210, 212 both prior to exercise onset and during exercise. There are approximately four to five heartbeats per respiratory cycle prior to exercise onset increasing to five to six beats per respiratory cycle during exercise.

FIGS. 2C and 2D respectively illustrate simulated cardio-respiratory data for a healthy heart exhibiting RSA and an unhealthy heart exhibiting no RSA. The cardio-respiratory data is plotted with phase angle of the respiratory cycle plotted on the horizontal axis (on a scale of 0 to 1 equivalent to 0 to 2π radians in the plots of FIGS. 2A and 2B) and R-wave deviation from the mean plotted on the vertical axis. Here, R-wave deviation refers to the deviation of R-R interval (or heartbeat period) from the mean R-R interval,

RSA can be clearly seen in FIG. 2C as a cycle in the R-wave deviation from a positive value to a negative value and back to a positive value. In contrast, FIG. 2D sees no variation in the R-wave deviation indicative of an absence of RSA.

Heart beats are shown on each plot of FIGS. 2C and 2D for multiple respiratory cycles and the data can be seen to form clusters 214. The clusters define a plurality of heartbeats occurring at the same respiratory phase angle over a number of respiratory cycles. In other words, the data illustrates CRPS for both the heart exhibiting RSA and the heart where RSA is absent. In both examples of FIGS. 2C and 2D the repeat sequence of phase angles occurs over more than one respiratory cycle, as evidenced by the large number of beats per respiratory cycle. For example, every other illustrated cluster 214 may correspond to a first respiratory cycle with the intervening clusters 214 corresponding to a second respiratory cycle, if the repeat sequence is over two respiratory cycles.

FIGS. 2A to 2D illustrate that both healthy and unhealthy hearts naturally tend towards exhibiting CRPS 202-210 regardless of the presence of RSA. FIG. 2A illustrates that CRPS is present in a healthy individual when the cardiorespiratory system is stressed during exercise, FIG. 2B illustrates that CRPS is present in an unhealthy cardiorespiratory system both at rest and during exercise and FIGS. 2C and 2D illustrate that CRPS is present in both the presence and absence of RSA.

Therefore, a heart may be considered as having three operating states: (i) unsynchronised with no CRPS and no RSA, particularly prevalent when the heart is under severe stress and/or there is an interruption in breathing; (ii) phase synchronised CRPS with no RSA, particularly prevalent when a person is performing an activity such as exercise; and (iii) phase synchronised CRPS with RSA, prevalent when a healthy heart is at rest or performing a moderate amount of activity relative to an underlying fitness. In various embodiments of the present disclosure, cardiorespiratory monitoring devices are provided which may monitor a health indication of a subject based on whether the cardiorespiratory data is indicative of state (i), (ii) or (iii). The monitoring device may also consider a demand on the heart (activity, etc.) relative to an underlying fitness and/or tissue health.

A first aspect of the present disclosure provides cardiorespiratory monitoring devices that monitor values of the two types of natural cardiorespiratory synchronisation in healthy individuals-RSA at rest (and low levels of activity relative to an individual's fitness level) and a continual tendency towards CRPS. The first aspect provides cardiorespiratory monitoring devices that can provide a health indication of a subject based on the RSA and CRPS values. Specifically, the device can determine: (i) a RSA value for the subject based on monitored cardiac data and respiratory data for the subject; and (ii) a CRPSvalue for the subject based on monitored cardiac data and respiratory data for the subject

The monitoring device (which may be referred to as the monitoring device or the device) receives sensor data comprising cardiac data and respiratory data. The sensor data may comprise data captured over an extended period of time such that it includes a plurality of respiratory cycles and an associated plurality of heart beats. The sensor data may be captured over a number of seconds, minutes, hours, days or weeks. The sensor data may be labelled with a time stamp such that the different sensor data can be aligned in time. In some examples, the device may include one or more sensors for capturing the sensor data. Capturing the sensor data with the device can ensure that the separate sensor data is captured with respect to the same time reference. The sensor data may include other types of physiological data including: pulse oximeter data, activity level data, for example accelerometer data, body temperature data, perspiration data, camera data, an adenosine triphosphate, ATP, level and an adrenaline level. Such data may also include time-stamps and the device may include a corresponding sensor accordingly.

In some examples, the device may determine the RSA value or the CRPS value dependent on a patient activity level indicated by the sensor data. For example, as described above in relation to FIG. 2A, CRPS may be more difficult to identify when a patient is at rest. Therefore, the device may determine the CRPS value based on a portion of the cardiac data and the respiratory data for which a patient activity level is greater than a CRPS threshold activity level. (The CRPS threshold level may correspond to moderate activity, such as a subject going about their daily activities, but not at rest). Similarly, as described in relation to FIG. 1A, RSA reduces during physical exertion. Therefore, the device may determine the RSA value based on a portion of the cardiac data and the respiratory data for which a patient activity level is less than a RSA threshold activity level. The RSA threshold activity level may correspond to moderate activity such as a subject going about their daily activities but not exercising.

In some examples the device may receive cardiac data and respiratory data having associated activity level data. For example, each heart beat, respiratory cycle or segments thereof, may include activity level data indicating an activity level of the subject when the data was captured. In some examples, the device may receive separate activity data captured by an activity sensor such as an accelerometer. In some examples, the device may determine the activity level based on the cardiac data and/or the respiratory data.

In some examples, the device may receive or determine activity data and: (i) determine a plurality of RSA values and a plurality of CRPS values during a period of physiological demand as indicated by the activity data; and (ii) determine the health indication based on the plurality of RSA values and the plurality of CRPS values. In some examples, the device may determine the health indication based on a variation in the plurality of RSA values and a variation in the plurality of CRPS values. For example, the variation in the plurality of RSA values may correspond to a degradation in the RSA value from the onset of exercise. The variation in the plurality of CRPS values may be compared to a threshold variation range to determine a duration of phase locking of the heart beats indicative of CRPS. The period of physiological demand may correspond to a period of exercise or a period of brain activities such as autonomic nervous system response. The period of exercise may be a period of exercise undertaken by the subject according to an exercise protocol. In some examples, the device may instruct the subject to exercise according to the exercise protocol.

A monitoring device according to the first aspect, can advantageously monitor cardiorespiratory synchronisation in a subject to provide additional insight into patient health. The device can monitor disease progression, damage to heart muscle (remodelling of the myocardium), cardiac pumping function or pacing device performance in order to improve quality of life outcomes for the subject of the monitoring device.

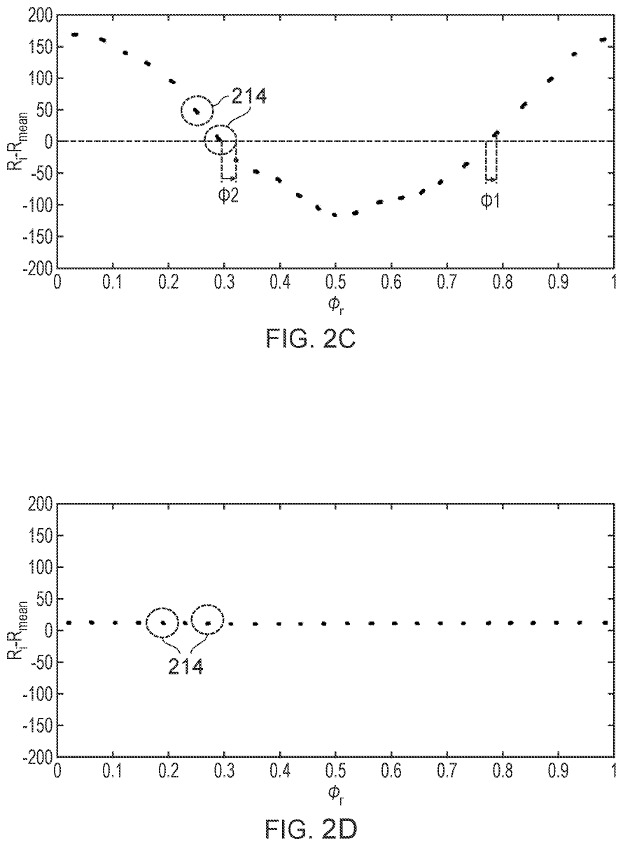

FIG. 3 illustrates a schematic block diagram of a monitoring system 300 comprising a monitoring device 302 according to one or more embodiments of the present disclosure. The monitoring system 300 comprises a monitoring device 302, sensors 304 and a user interface 306. The monitoring system 300 is an electronic device for monitoring both: (i) the biological RSA process present in a healthy individual at rest; and (ii) the biological CRPS process that all hearts tend towards in the absence of respiratory or cardiac interruption, and outputting a health indication accordingly.

The sensors 304 may comprise a respiratory sensor for providing respiratory data, and a cardiac sensor for providing cardiac data, as sensor data to a controller 308. The respiratory sensor may comprise an electromyography sensor or a chest impedance sensor, for example. The respiratory sensor may be configured to sense a signal indicative of respiration of the subject. The cardiac sensor may comprise one or more electrodes. The cardiac sensor may comprise an electrocardiogram (ECG) sensor or other known cardiac sensors. The sensors 304 may further comprise an activity sensor for providing activity data relating to the subject.

The respiratory data may comprise minute ventilation data. The controller may be configured to determine a minute ventilation parameter based on the minute ventilation data. Minute ventilation (VE) is defined as the volume of gas inhaled (inhaled minute ventilation) or exhaled (exhaled minute ventilation) from a person's lungs per minute. Minute ventilation can be written as:

$$VE = TV \times f$$

where, TV=tidal volume
f=frequency of respiration

Tidal volume is a measure of the volume of air displaced between normal inhalation and exhalation without extra effort. Minute ventilation (VE) is proportional to respiratory effort and the frequency of respiration. The controller 308 may determine the activity level based on the minute ventilation parameter.

Any of the sensors 304 may be provided by conventional sensors used in the art (such as those described in the background section above) and may include mechanisms or adhesives for coupling the sensors 304 to the subject. The monitoring device 302 may be configured to communicate with the sensors 304 via a wireless or wired communications link. For example, a near-field communications link could operate through tissue of the patient so that one of i) the monitoring device 302 and ii) one or more of the sensors 304 is provided internally and the other is provided externally.

The one or more cardiac sensor electrodes may comprise a mechanism or adhesive for attaching the one or more electrodes to the subject.

The monitoring device 302 comprises a controller 308 and an output signal generator 310. The controller 308 may comprise one or more processors. The controller 308 can calculate the RSA and CRPS values and determine the health indication of the subject. The controller can provide the health indication to the output signal generator 310 for conveying to an external entity such as the user interface 306.

The controller 308 is configured to receive sensor data including the cardiac data and the respiratory data from the sensors 304 at a sensor signal input 312. The controller 308 is configured to determine the RSA value and the CRPS value based on the sensor data and provide the health indication for the patient in accordance with the RSA value and the CRPS value. The controller 308 may provide the health indication to the output signal generator 310 which can relay the health indication to the patient or a clinician via the user interface 306. For example, the output signal generator may generate a visual, audible and/or tactile stimulus to convey the health indication to the patient. In some examples, the output signal generator 310 may form part of the controller 308 and the controller may provide the health indication directly to the user interface 306. In some examples, the controller 308 or signal generator 310 may output the health indication to a monitoring centre and/or a networked database such as an electronic health record.

The cardiac sensor and or the device 302 may be implantable. For example, the device 302 may have a housing, which may be biologically inactive, or non-reactive. The housing may have dimensions of 10 cm or less by 5 cm or less, for example. The device 302 may include a power source to power the device 302.

The controller 308 may be provided by hardware, software or a combination of hardware and software. In one example, the controller 308 may be provided as software modules that are executed by hardware, for example.

According to the first aspect the controller 308 of the monitoring device 302 is configured to receive sensor data comprising respiratory data and cardiac data. The controller 308 may receive the sensor data from the one or more sensors 304. The controller 308 is configured to determine a RSA value and a CRPS value based on the sensor data and output the health indication in accordance with the RSA value and the CRPS value.

The device 302 may process the respiratory data to identify a one or more respiratory cycles, including inspiration and expiration phases of each respiratory cycle. For example, the device 302 may identify respiratory cycles using zero-crossings of the transthoracic impedance (and use the polarity of the signal either side of the zero-crossings to identify inspiration/expiration phase). The device 302 may associate time points of the respiratory data with a phase angle corresponding to the phase of the respiratory cycle at a particular time point.

The device 302 may identify a plurality of heart beats from the cardiac data. For example, the device may identify a particular peak (such as the R-wave peak) as a heart beat.

As noted above, the device 302 may align the respiratory data and cardiac data using time stamps of each data set.

To determine the RSA value, the device 302 may first segment the cardiac data into: (i) one or more inspiration sequences of heart beats occurring in an inspiration phase of a respiratory cycle; and (ii) one or more expiration sequences of heart beats occurring in an expiration phase of a respiratory cycle. The device 302 may identify whether a heart beat is in an inspiration or expiration phase of a heart beat based on a value, phase or phase angle of the respiratory data at the time stamp of the heart beat. The device 302 may determine an inspiration heart rate based on the one or more inspiration sequences and an expiration heart rate based on the one or more expiration sequences. The device 302 may determine the RSA value based on the inspiration heart rate and the expiration heart rate.

In some examples, the device 302 may determine an inspiration heart rate, expiration heart rate and RSA value for each pair of inspiration and expiration sequences corresponding to the same respiratory cycle. In some examples, the device may determine the inspiration heart rate, expiration heart rate and RSA value based on an average of a plurality of pairs of inspiration and expiration sequences corresponding to the same respiratory cycle, for example, 2, 3, 4, 5 or more pairs.

In some examples, the device 302 may determine the RSA value based on a difference between the inspiration rate and the expiration rate. For example, the device 302 may determine the RSA value as the difference between, or a ratio of, the inspiration and expiration rates. The device may determine the inspiration and expiration rates as a number of beats per time period (e.g. seconds, minutes etc) or as a number of beats per respective inspiration or expiration phase. The device may determine the rates based on an average period between the heart beats in a particular sequence or in a plurality of sequences.

To determine the CRPS value, the device 302 may associate each heart beat with an identified respiratory cycle and a phase angle of the respiratory cycle, to provide labelled cardiorespiratory data. The device 302 may associate a heartbeat with a phase angle based on the phase angle of the respiratory data at the time stamp of the heartbeat. The device 302 may determine the CRPS value from the labelled cardiorespiratory data. The device may determine the CRPS value based on a variance in respiratory phase angles over a plurality of respiratory cycles. For example, the device 302 may determine labelled cardiorespiratory data including a plurality of respiratory cycles, for example, 2, 3, 4, 5, 10 or more respiratory cycles. The labelled cardiorespiratory data may be similar to the data of FIG. 2D in that the heart beats form clusters having substantially the same phase angle. The device 302 may identify clusters having substantially the same phase angle. Here, substantially the same phase angle may be defined as a variation in phase angle between corresponding heartbeats in respiratory cycles being less than a CRPS threshold, for example 0.1, 0.2, 0.3, 0.4 or 0.5 radians. The device may determine the CRPS value based on: (i) the number of clusters; (ii) the number of clusters relative to the number of heart beats per respiratory cycle; (iii) a clustering factor identifying the clustering or lack of clustering in the labelled respiratory data; (iv) the variance or distribution of the heart beats in each cluster; (v) the (average) phase angles of the clusters; and/or (vi) a phase angle period between adjacent heartbeats/clusters. The device 302 may incrementally increase the number of respiratory cycles in the labelled respiratory data to determine a duration of phase locking, that is the number of respiratory cycles for which the heart beats remain in the same set of clusters.

In some examples of CRPS, the heart beats may occur at the same phase angle in consecutive respiratory cycles (see for example the plateaus 202, 204, 206, 208, 210, 212 in FIGS. 2A and 2B). However, in other examples of CRPS the phase angle repeat sequence may occur over a plurality of respiratory cycles (such as in FIGS. 2C and 2D). As a simplified example with no RSA present, for a baseline heartbeats per respiratory cycle of 5.5 beats per respiratory cycle, the device may detect clusters at average phase angles of 0, 2n/11, 4n/11, 6n/11, 8n/11, 10n/11, n/11, 3n/11, 5n/11, 7n/11 and 9n/11 over adjacent sets of two respiratory cycles. The cluster analysis can account for phase angle repeat sequences over more than one respiratory cycle. The device 302 can identify that there is no CRPS based on the clustering factor being less than a clustering threshold. The device 302 can identify that CRPS is present based on the clustering factor exceeding the clustering threshold. The device 302 can identify the number of respiratory cycles over which the set of phase angles repeat based on the number of clusters relative to the number of heart beats per respiratory cycle.

In some examples, the device 302 may determine the CRPS value based on a variance in the number of heart beats per respiratory cycle or per inspiration and expiration phase, or based on a variance of the period between adjacent heart beats. Such a CRPS value may provide a simpler analysis of CRPS than the cluster approach.

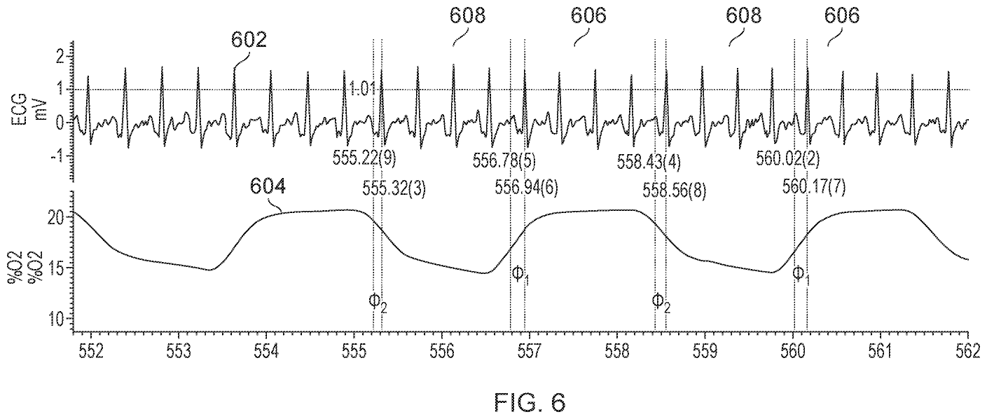

FIG. 6 illustrates a further example of determining a CRPS value. The figure illustrates cardiac data 602 and respiratory data 604 plotted against time (in seconds) for a subject. Inspiration phases 606 and expiration phases are shown for the subject. The data is indicative of CRPS in the subject with no RSA as evidenced by the number of heart beats being the same during inspiration and expiration (4 beats/inspiration and 4 beats/expiration). An inspiration onset phase angle, Φ1, is the first phase angle in the inspiration phase 606. The inspiration onset phase angle, Φ1, may comprise the phase difference between the onset of inspiration and the next heartbeat. An expiration onset phase angle, Φ2, is the first phase angle in the expiration phase 608. The expiration onset phase angle, Φ2, may comprise the phase difference between the onset of expiration and the next heartbeat. Φ1 is the phase locking of next heartbeat to inspiration phase and Φ2 is the phase locking of heartbeat to expiration phase. Φ1 and Φ2 may or may not be the same. The frequency of heartbeats (heartrate) during inspiration is the same as during expiration indicating no RSA. The figure shows constant values of the inspiration and expiration onset phase values, Φ1 and 2, for two consecutive breath cycles, within margin of errors. Therefore, the device may determine the CRPS value using the inspiration onset phase angle, Φ1, and/or the expiration onset phase angle, Φ2. For example, the device may determine the CRPS value based on a variance of the inspiration onset phase angle, Φ1, and/or the expiration onset phase angle, Φ2.

Although FIG. 6 illustrates cardiorespiratory data with no RSA, the device can determine the same values of inspiration onset phase angle, Φ1, and/or the expiration onset phase angle, Φ2, for cardiac and respiration data in which RSA is present (as illustrated by the annotations of Φ1 and Φ2 in FIG. 2C). The inspiration onset phase angle, Φ1, and the expiration onset phase angle, Φ2, can be different when RSA is present. The device may determine the CRPS value, or a phase locking value (or phase synchronisation value) of cardiorespiratory data in which RSA is present, based on the inspiration onset phase angle, Φ1, and/or the expiration onset phase angle, Φ2.

The device 302 may determine a RSA health factor for the subject by comparing the RSA value to one or more RSA reference values. The device 302 may determine a CRPS health factor for the subject by comparing the CRPS value (or plurality of values) to one or more CRPS reference values. The one or more reference values (for either or both of CRPS and RSA) may comprise predetermined RSA values based on a reference population of subjects. The one or more reference values may be obtained by subjecting the population to the same exercise protocol, for example a protocol comprising periods of rest and periods of activity of monitored exercise activity, e.g., on an exercise machine. The one or more reference values may comprise sub-sets of reference values categorised according to underlying health conditions and/or demographics, such age, sex, ethnicity etc. The one or more reference values may be stored in a look-up table that maps RSA values to health indication. In some examples, the one or more reference values may comprise one or more thresholds defining different values of the CRPS/RSA health factor. By using one or more reference values, the device can compare the data with ‘healthy values’ estimated by averaging of the healthy values in a number of healthy subjects and ‘unhealthy values’ estimated by average of unhealthy values in a number of unhealthy subjects. The reference population may comprise a cohort of people subjected to a standardised level of work from which their individual health indication (metric) can be derived to provide the predefined reference values. The reference values provide a metric for use as an indicator of healthy, unhealthy, highly fit, moderately fit and unfit within the cohort. The metric can be used as an indicator of improving or declining health of subjects using the device in response to their lifestyle changes, changes in physical abilities etc.

The device 302 may determine the health indication based on the RSA health factor and the CRPS health factor. For example, the device 302 may determine the health indication by matching the RSA health factor and the CRPS health factor to a health indication using a look up table. It will be appreciated that the device 302 may bypass determining the RSA and/or CRPS health factor step and simply match the RSA value and the CRPS value to a health indication using a look up table of health indication values for RSA and CRPS value pairs. The look up table may be defined using predefined reference values for a sample population as described above for the RSA and CRPS factors.

In examples, where the device determines a plurality of RSA and CRPS values the device may compare each value, or each value pair against corresponding reference values to determine a health indication for each RSA/CRPS value pair. In this way, the device 302 can provide a “live” or dynamic value of health indication. In other examples, the device 302 may average, sum or otherwise process the plurality of RSA/VRPS values (for example taking the area under a curve) to determine an effective RSA/CRPS value for comparison against the one or more reference values. The latter could be particularly applicable in examples when a plurality of RSA/CRPS values are captured during a period of physiological demand and the health indication is based on a variation of RSA and CRPS during the demand compared against one or more reference variations of the sample population.

In some examples, the device may determine the RSA factor, the CRPS factor and/or the health indication based on one or more of the other types of physiological data, described above. For example, the device may determine a health indication based on the RSA factor, the CRPS factor and blood oxygen level.

The device 302 may output the health indication values (and optionally the RSA and CRPS values) to a user interface 310 of the system 300 to inform the subject of the health indication. The device 302 may also output the health indication to a clinician device, for example, for display on a user interface of the clinician device and/or for storage in a memory of the clinician device for subsequent review. The device 302 may also output the health indication to an emergency responder device if the health indication indicates significant cardiorespiratory stress. The device 302 may also output the health indication to a remote monitoring centre. In some examples, the device 302 may output the RSA value, the CRPS value and/or the health indication value to a pacemaker device to implement corrective pace making. In some examples, the device may output the health indication to an electro stimulating system, such as a peripheral stimulation device, to implement peripheral stimulation, for example transcutaneous stimulation. Transcutaneous stimulation is advantageously less invasive than a pacing device. The health indication signal may comprise an information signal (for example for output to user interfaces or memory. The health indication signal may comprise a control signal for controlling intervention devices such as the pacemaker device or the peripheral stimulation device.

In some examples, the device 302 may output therapeutic instructions to the subject if the health indication is representative of cardiorespiratory stress in the subject. For example, the device 302 may output therapeutic instructions to a speaker, screen and/or haptic interface of a subject user interface 310. The subject user interface 310 may be part of the user device 302 or be separate to but associated with the device 302, for example the user interface of a personal user device such as a tablet or smartphone. The therapeutic instructions may comprise one or more of: breathing instructions; activity instructions, such as an instruction to rest or to undertake meditation or wellbeing activities; and an instruction to seek immediate medical attention. In this way, the device can provide an aid to achieve improved metric (health indication) outcome for sensory/haptic indication and guidance. The breathing techniques can include guided respiratory techniques optionally with physical exercises (example—moving hands and legs in coordination with respiration) to aid and improve the cardiorespiratory coupling as indicated by the metric.

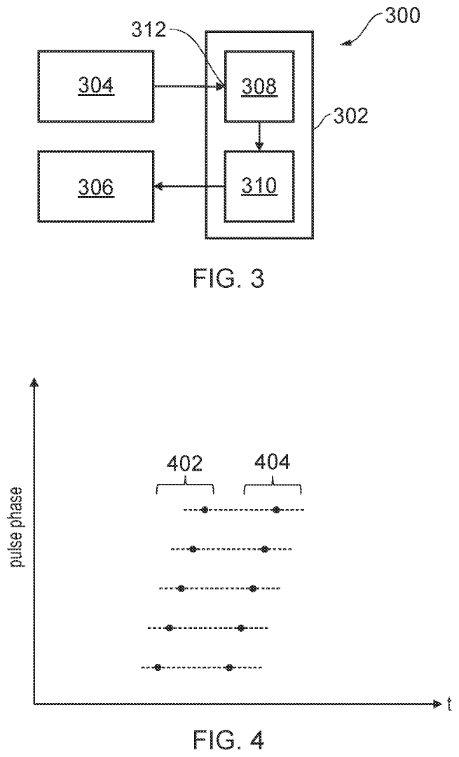

FIG. 4 illustrates a plot of heart beat sequences in CRPS mode against time for two successive respiratory cycles 402, 404. The plot is a synchrogram plot of the type described previously with reference to FIGS. 2A and 2B, in which heartbeat instances, in time, on the x-axis is plotted with respect to a respiratory phase on the y-axis. In this example, each respiratory cycle contains five heartbeats. Each heartbeat in the first respiratory cycle 402 is in phase with a respective heartbeat in the subsequent, second respiratory cycle 404. Each heartbeat occurs at a particular time with respect to the onset of its respiratory cycle (that is, after a period of time has elapsed). The corresponding heartbeat of the subsequent respiratory cycle occurs at the same time with respect to the onset of its respiratory cycle. CRPS is evident with a repeat sequence over each respiratory cycle.

In one or more examples, the device 302 may determine a baseline health indication of the subject. For example, sensor data including cardiological data and respiration data may be monitored for a patient undergoing an exercise protocol including varying intensity levels of exercise to determine a set of baseline RSA, CRPS and health indication values. The device 302 may store the set of baseline data. In operation, the device may compare RSA values, CRPS values and or health indication values against the base line values to determine a personalised health indication. In other words, the baseline values may form the reference values described above.

The cardiorespiratory system may be functioning under a temporary increased stress when the patient physically exerts themselves, such as during exercise or lifting a heavy load. An increased stress may also be present when the patient is mentally stressed or following ingestion. Cardiorespiratory stress may also be dependent on one or more of: ambient temperature, O2 content (thin air), body position, dehydration, fever, starvation, noise, cold exposure, haemorrhage (menstrual cycle), psychological factors, immunological factors, pain and disease. The system may comprise one or more sensors to determine a level of one or more of these stress factors.

The sensor data may comprise other data for indicating the patient activity level. For example, the controller 308 may receive sensor data comprising one or more of: cardiological data, pulse oximeter data, accelerometer data, body temperature data, perspiration data, camera data, adenosine triphosphate, ATP, levels, adrenaline levels or other data suitable for determining an activity level as known in the art. The other sensor data may be received from one or more corresponding sensors as are known in the art, such as an accelerometer, a pulse oximeter, a thermometer or a camera. The sensors may form part of the cardiorespiratory monitoring system 300 or may be external to the system.

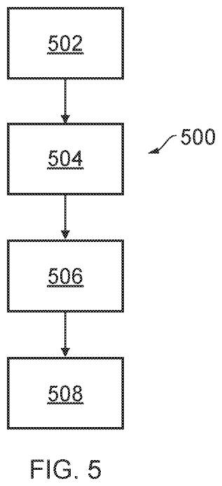

FIG. 5 illustrates a method 500 for providing a health indication signal, which may be performed by the device described previously with reference to FIG. 3.

The method 500 comprises:
receiving 502 sensor data comprising cardiac data and respiratory data for a subject;
determining 504 a RSA value based on the cardiac data and the respiratory data;
determining 506 a CRPS value based on the cardiac data and the respiratory data; and
outputting 508 the health indication based on the RSA value and the CRPS value.

A second aspect of the present disclosure provides cardiorespiratory monitoring devices that monitors cardiorespiratory synchronisation data (such as RSA value or CRPS values) during a period of physiological demand to determine a health indication.

The device of the second aspect receives sensor data comprising cardiac data and respiratory data for a subject. The device determines cardiorespiratory synchronisation data (e.g. one or more RSA values and/or one or more CRPS values) based on the cardiac data and the respiratory data. The device determines a cardiorespiratory coupling factor based on a response of the cardiorespiratory synchronisation data to a period of physiological demand, as indicated by the sensor data. For example, the device may determine the cardiorespiratory coupling factor based on a plurality of cardiorespiratory synchronisation values captured during the period of physiological demand. The device outputs the health indication for the subject based on the cardiorespiratory coupling factor.

In some examples, the sensor data may comprise activity data. In some examples, the device can determine the activity data from the cardiac data and/or the respiratory data. The device can identify the period of physiological demand based on the activity data.

In some examples, the device can determine the cardiorespiratory synchronisation data by determining a plurality of RSA values during the period of physiological demand. The RSA values may be determined in the same manner as described above for the first aspect.

The device may determine the cardiorespiratory coupling factor based on a variation in the plurality of RSA values during the period of physiological demand. For example, the variation in the plurality of RSA values may correspond to a degradation in the RSA value from the onset of exercise.

In some examples, the device can determine the cardiorespiratory synchronisation data by determining a plurality of CRPS values during the period of physiological demand. The CRPS values may be determined in the same manner as described above for the first aspect. The device may determine the cardiorespiratory coupling factor based on a variation in the plurality of CRPS values during the period of physiological demand. The variation in the plurality of CRPS values may be compared to a threshold variation range to determine a duration of phase locking of the heart beats indicative of CRPS.

The period of physiological demand may correspond to a period of exercise or a period of brain activities such as autonomic nervous system response. The period of exercise may be a period of exercise undertaken by the subject according to an exercise protocol.

The coupling factor can reflect the responsiveness of the subject's heart to physiological demands including but not limited to physical constraints, brain activities such as autonomic nervous system response. The coupling factor can be high in a healthy cardiorespiratory system and decrease with cardiorespiratory illness, ageing and other health related factors.

In some examples, the health indication may comprise the cardiorespiratory coupling factor. In some examples, the device may determine the health indication by comparing the cardiorespiratory coupling factor to one or more reference values. For example, the device may determine the health indication by matching the cardiorespiratory coupling factor to the health indication using a look up table of reference values. The one or more reference values may comprise pre-defined relationships between cardiorespiratory synchronisation data, cardiorespiratory coupling factor and health indication determined using a sample population in the same way as described above in relation to the first aspect. The predefined relationships may have been determined by subjecting the sample population to the same exercise protocol. The period of physiological demand may comprise the subject exercising according to the same exercise protocol. In some examples, the device may instruct the subject to follow the exercise protocol.

The device of the second aspect may be implemented as the device of FIG. 3.

It will be appreciated that the device of the second aspect may include a number of features of the first aspect described above. For example, the device of the second aspect may: determine RSA values; determine CRPS values; utilise other sensor data types; determine values of coupling factors or health indication using reference values; and output the health indication in substantially the same was as described for the first aspect. In other words, the second aspect may be considered the same as the first aspect with the exception that the second aspect determines a health indication based on a response of cardiorespiratory synchronisation data to a period of physiological demand.

A controller may be configured to perform any method described herein, including the method 500 as a computer-implementable method. The controller may comprise one or more processors and memory, the memory comprising computer program code configure to cause the processor to perform any method described herein.

There may be provided a computer program, which when run on a computer, causes the computer to configure any apparatus, including a controller for a cardiorespiratory monitoring device to perform any method disclosed herein. The computer program may be a software implementation. The computer may comprise appropriate hardware, including one or more processors and memory that are configured to perform the method defined by the computer program.

According to a further aspect of the disclosure there is provided a computer readable storage medium comprising computer program code configure to cause a processor to perform any computer-implementable method described herein. The computer readable storage medium may be a non-transitory computer readable storage medium. The computer program may be provided on a computer readable medium, which may be a physical computer readable medium such as a disc or a memory device, or may be embodied as a transient signal. Such a transient signal may be a network download, including an internet download.

1. A cardiorespiratory monitoring device, configured to:
receive sensor data comprising cardiac data and respiratory data for a subject;
determine a respiratory sinus arrhythmia, RSA, value based on the cardiac data and the respiratory data;
determine a cardio-respiratory phase synchronisation, CRPS, value based on the cardiac data and the respiratory data; and
output a health indication for the subject based on the RSA value and the CRPS value.

2. The device of claim 1, configured to:
determine a subject activity level based on the sensor data; and
determine the RSA value based on at least a portion of the cardiac data and the respiratory data for which the subject activity level is less than a RSA threshold activity level; and/or
determine the CRPS value based on at least a portion of the cardiac data and the respiratory data for which the subject activity level is greater than a CRPS threshold activity level.

3. The device of claim 1, wherein the device is configured to:
receive activity data;
determine a plurality of RSA values and a plurality of CRPS values during a period of physiological demand as indicated by the activity data; and
determine the health indication based on the plurality of RSA values and the plurality of CRPS values.

4. The device of claim 3, wherein the device is configured to determine the health indication based on:
a variation in the RSA values during the period of physiological demand; and
a variation in the CRPS values during the period of physiological demand.

5. The device of claim 1, wherein the device is configured to:
identify a plurality of respiratory cycles from the respiratory data;
identify a plurality of heart beats from the cardiac data;
associate each heart beat with a respiratory cycle and a respiratory phase angle of the respiratory cycle to provide labelled cardiorespiratory data;
determine the CRPS value from the labelled cardiorespiratory data.

6. The device of claim 5, wherein the device is configured to:
determine the CRPS value based on a variance in respiratory phase angles over the plurality of respiratory cycles.

7. The device of claim 1, wherein the device is configured to:
identify one or more respiratory cycles from the respiratory data;
identify a plurality of heartbeats from the cardiac data;
identify one or more inspiration sequences of heart beats in inspiration phases of the plurality of respiratory cycles and one or more of expiration sequences of heart beats in expiration phases of the plurality of respiratory cycles; and
determine the RSA value based on an inspiration heart rate of the one or more inspiration sequences and an expiration heart rate of the one or more expiration sequences.

8. The device of claim 7, wherein the device is configured to determine the RSA value as a ratio of the inspiration heart rate to the expiration heart rate.

9. The device of claim 1, wherein the device is configured to:
determine a RSA health factor for the subject by comparing the RSA value to one or more RSA reference values;
determine a CRPS health factor for the subject by comparing the CRPS value to one or more CRPS reference values; and
output the health indication based on the RSA health factor and the CRPS health factor.

10. The device of claim 1, wherein the device is configured to determine the health indication using a look up table and the CRPS value and the RSA value.

11. The device of claim 1, wherein the sensor data comprises a plurality of sensor readings over a time window and wherein the device is configured to determine a plurality of RSA values and a plurality of CRPS values and output the health indication based on the plurality of RSA values and the plurality of CRPS values.

12. The device of claim 11, wherein the device is configured to:
determine a RSA health factor for the subject by comparing the plurality of RSA values to one or more RSA reference values;
determine a CRPS health factor for the subject by comparing the plurality of CRPS values to one or more CRPS reference values; and
output the health indication based on the RSA health factor and the CRPS health factor.

13. The device of claim 1, wherein the device is configured to output the health indication to one or more of:
a subject user interface;
a clinician device;
a pacemaker device;
a peripheral stimulation device;
an emergency responder device; and
a remote monitoring centre.

14. The device of claim 1, wherein the device is configured to output therapeutic instructions to a subject user interface if the health indication is representative of cardiorespiratory stress in the subject.

15. The device of claim 14, wherein the therapeutic instructions comprise one or more of:
breathing instructions;
activity instructions; and
an instruction to seek immediate medical attention.

16. A cardiorespiratory monitoring system comprising:
the device of claim 1; and
one or more sensors configured to provide the sensor data.

17. The cardiorespiratory monitoring system of claim 16, further comprising a patient user interface for displaying the health indication.

18. A method for providing a health indication, the method comprising:
receiving sensor data comprising cardiac data and respiratory data for a subject;
determining a respiratory sinus arrhythmia, RSA, value based on the cardiac data and the respiratory data;
determining a cardio-respiratory phase synchronisation, CRPS, value based on the cardiac data and the respiratory data; and
outputting a health indication for the subject based on the RSA value and the CRPS value.

19. (canceled)

20. A cardiorespiratory monitoring device, configured to:
receive sensor data comprising cardiac data and respiratory data for a subject;
determine cardiorespiratory synchronisation data based on the cardiac data and the respiratory data;
determine a cardiorespiratory coupling factor based on a response of the cardiorespiratory synchronisation data to a period of physiological demand as indicated by the sensor data; and
output a health indication for the subject based on the cardiorespiratory coupling factor.

21. The device of claim 20, wherein the device is configured to determine the cardiorespiratory synchronisation data by determining one or more of:
a plurality of respiratory sinus arrhythmia, RSA, values based on the cardiac data and the respiratory data; or
a plurality of cardio-respiratory phase synchronisation, CRPS, values based on the cardiac data and the respiratory data.

22.-25. (canceled)
